(19) United States
(12) Patent Application Publication
(10) Pub. No.: US 2003/0013668 A1
(43) Pub. Date: Jan. 16, 2003

(54) ANTISENSE OLIGONUCLEOTIDES TARGETED TO IL-15

(76) Inventors: DANGE VEERAPANANE PH.D, SAN DIEGO, CA (US); SHOJI HAMANAKA, YOKOSUKA (JP); HIROYUKI KUBO, CARLSBAD, CA (US); IWAO NOZAWA, CARLSBAD, CA (US)

Correspondence Address: FISH & RICHARDSON, PC, 4350 LA JOLLA VILLAGE DRIVE, SUITE 500, SAN DIEGO, CA, 92122, US

(21) Appl. No.: 09/349,216

(22) Filed: Jul. 7, 1999

Related U.S. Application Data
(60) Provisional application No. 60/091,873, filed on Jul. 7, 1998.

(51) Int. Cl. A61K 48/00; C07H 21/04

(52) U.S. Cl. 514/44; 536/23.5

(57) ABSTRACT

The invention features antisense oligonucleotide molecules that specifically bind polynucleotides encoding IL-15. The present invention provides antisense oligonucleotides capable of inhibiting IL-15 expression, and methods of use thereof to reduce activity of IL-15 in tissues in order to treat diseases such as rheumatoid arthritis, inflammatory bowel disease, multiple sclerosis, chronic liver disease, ulcerative colitis and cell proliferative disorders.

This application claims priority from U.S. Provisional Application Ser. No. 60/091,873 filed Jul. 7, 1998, the disclosure of which is incorporated herein by reference.

### FIELD OF THE INVENTION

This invention relates generally to the field of therapeutic compositions and more specifically to antisense oligonucleotides that bind to interleukin-15 (IL-15 ) polynucleotides and methods of treatment for diseases associated with IL-15.

### BACKGOUND OF THE INVENTION

There are a number of diseases known in humans that affect various tissues, including the joints, and particularly the synovium. These include synovial sarcomas, osteoarthritis, bacterial and fungal infections, and inflammatory, autoimmune, and hemorrhagic diseases. Combined, they are a cause of great pain and suffering in the population, with little effective therapy apart from symptomatic treatment with analgesics and anti-inflammatory drugs (reviewed by Gardner, 1994 J. Anat. 184:465-76).

Rheumatoid arthritis (RA) affects one percent of the population worldwide. There is significant immunological activity within the synovium during the course of the disease. It is believed that this reactivity provides an intense stimulus to the synovial lining cells, which then undergo a transformation into an invasive pannus that brings about joint erosion through the release of destructive mediators. The release of cytokines, proteases, and reactive oxygen intermediates, have all been implicated in the disease pathology. The initiating factor is unknown, but might be an infection, trauma, bacterial infection, or autoreactivity. For example, there is increased risk of developing rheumatoid arthritis for persons having the HLA-Dw4 allele.

Chronic rheumatoid arthritis is characterized by infiltration of the normally relatively acellular synovial membrane by macrophages, T cells, and plasma cells, and with the presence of activated fibroblast-like synoviocytes (Duke, O., et al, 1982, Clin. Exp. Immunol. 49:22-30). There are several reports in the literature which document the presence the cytokines of macrophage derivation including IL-1, IL-1μ, IL-6, IL-8, IL-15, GM-CSF, and TNF-α in synovial proliferation. Cytokines associated with T cell activation such as IFN-γ, and IL-2 have also been detected in rheumatoid arthritis. The role of various cytokines and proteases in rheumatoid arthritis is discussed in Feldmann et al., 1994 Circ. Shock 43:179-84; and Testa et al., 1994 Clin. Orthop. 308:79-84. Additionally, a number of other disease states have been associated with an increased expression of interleukin-15 (IL-15). Such diseases include inflammatory bowel disease, multiple sclerosis, chronic liver disease, ulcerative colitis and certain cell proliferative disorders (Sakai et al., 1998 Gastroenterology, 114(6):1237-1243; Kivisakk et al., 1998 Clin Exp Immunol, 111(1): 193-197; Kacani et al., 1997 Clin Exp Immunol, 108(1):14-18; and Kirman et al., 1996 Am J Gastroenterol, 91(9):1789-1794). Therapies directed at T cells, such as cyclosporin A and monoclonal antibodies against T-cell surface antigens, can produce significant clinical improvement. However, additional therapies are still needed.

### SUMMARY OF THE INVENTION

This invention relates to antisense oligonucleotides that bind to polynucleotides encoding IL-15, thus preventing production of an IL-15 polypeptide. The present invention provides antisense oligonucleotides that inhibit IL-15 expression, and use thereof to reduce activity of IL-15 in tissues, in order to treat diseases such as rheumatoid arthritis, inflammatory bowel disease, multiple sclerosis, chronic liver disease, ulcerative colitis and cell proliferative disorders. The invention features use of antisense oligonucleotides to treat such diseases by inhibiting the synthesis of IL-15 and preventing the recruitment and activation of macrophages.

The invention features antisense oligonucleotide molecules that specifically bind polynucleotides encoding IL-15. In a preferred embodiment the antisense oligonucleotides bind mRNA encoding IL-15. In another embodiment, the antisense oligonucleotides are about 8 to about 40 nucleic acids in length and can be either DNA or RNA. The antisense oligonucleotides may be chemically modified.

In another embodiment, the invention features a method for suppressing IL-15 production in a cell by administering to the cell an amount of antisense oligonucleotide molecules sufficient to specifically bind polynucleotides encoding IL-15, thereby suppressing IL-15 levels. In another aspect, the invention features a method for treating a subject having or at risk of having an IL-15-associated disorder, by administering to the subject an effective amount of antisense oligonucleotide which specifically binds mRNA encoding IL-15. The IL-15 disorder may be an inflammatory disorder, for example. In a particular embodiment, the disorder is rheumatoid arthritis.

In still another aspect, the invention provides a pharmaceutical composition for treatment of a disorder associated with IL-15. The composition comprises an antisense oligonucleotides of the invention either alone, or in combination with other antisense molecules or pharmaceutical agents.

The invention provides several advantages. For example, the antisense oligonucleotides of the invention are specific for IL-15 polynucleotides. A further advantage of the present invention is that the antisense oligonucleotide molecules can be delivered exogenously or can be expressed from DNA or RNA vectors that are delivered to specific cells. In a preferred embodiment the antisense oligonucleotides are provided by transcription of a recombinant DNA sequence. The recombinant DNA sequence may be in a plasmid or viral vector.

In yet another embodiment, a method of monitoring the effectiveness of suppressing IL-15 expression after administering a therapeutically effective amount of the antisense oligonucleotide is provided, the method comprises detecting IL-15 levels before and after the antisense therapy.

Other features and advantages of the invention will be apparent from the following detailed description, and from the claims.

### BRIEF DESCRIPTION OF THE DRAWINGS

FIG. 1 shows the cDNA sequence of human IL-15 (SEQ ID NO: 9) (GenBank U14407).

FIG. 2 shows the antisense oligonucleotides of the invention (SEQ ID NOs: 1-8 and 10-16).

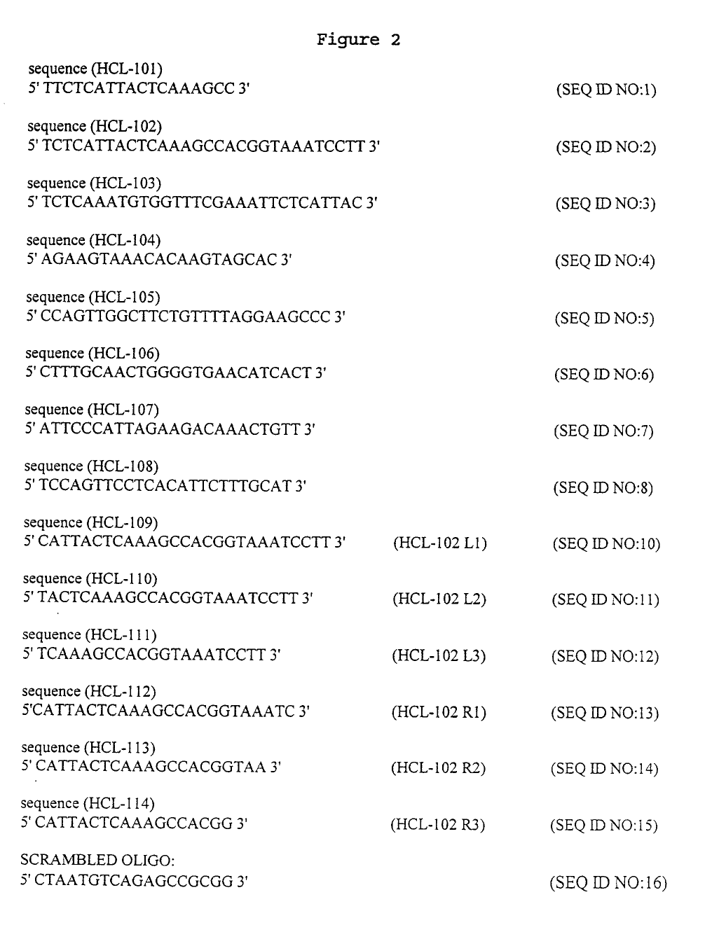

FIG. 3 is a line graph showing the effects of increasing concentrations of antisense oligonucleotide HCL-101 (SEQ ID NO: 1) on IL-15 synthesis in CV-1/EBNA cells.

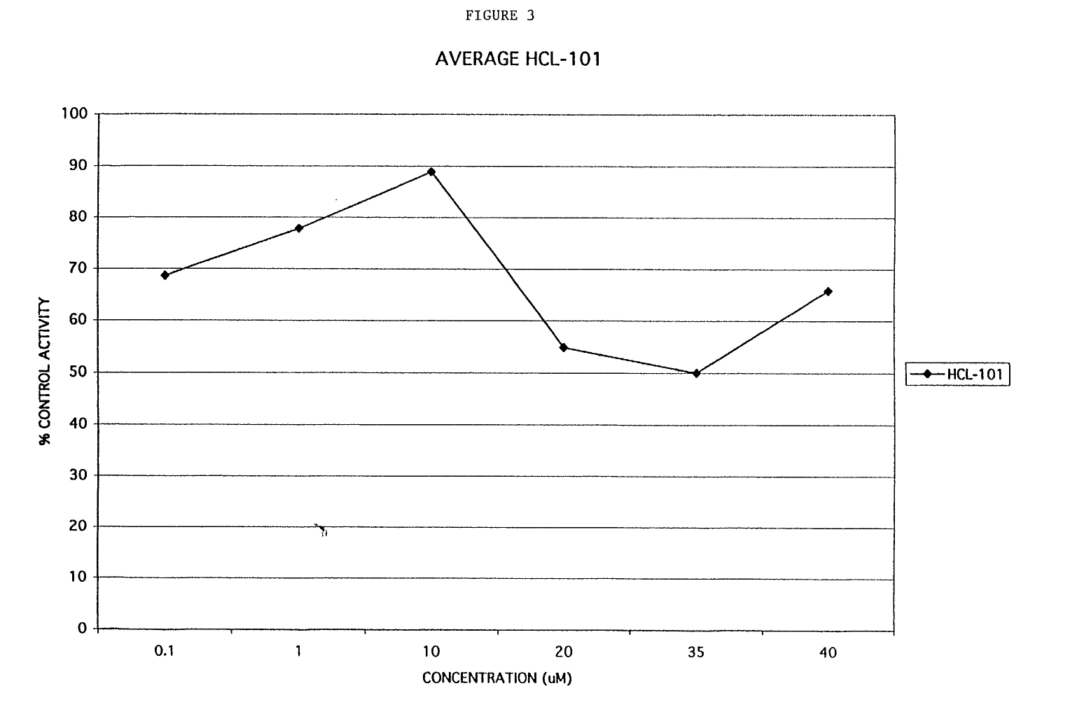

FIG. 4 is a line graph showing the effects of increasing concentrations of antisense oligonucleotide HCL-102 (SEQ ID NO: 2) on IL-15 synthesis in CV-1/EBNA cells.

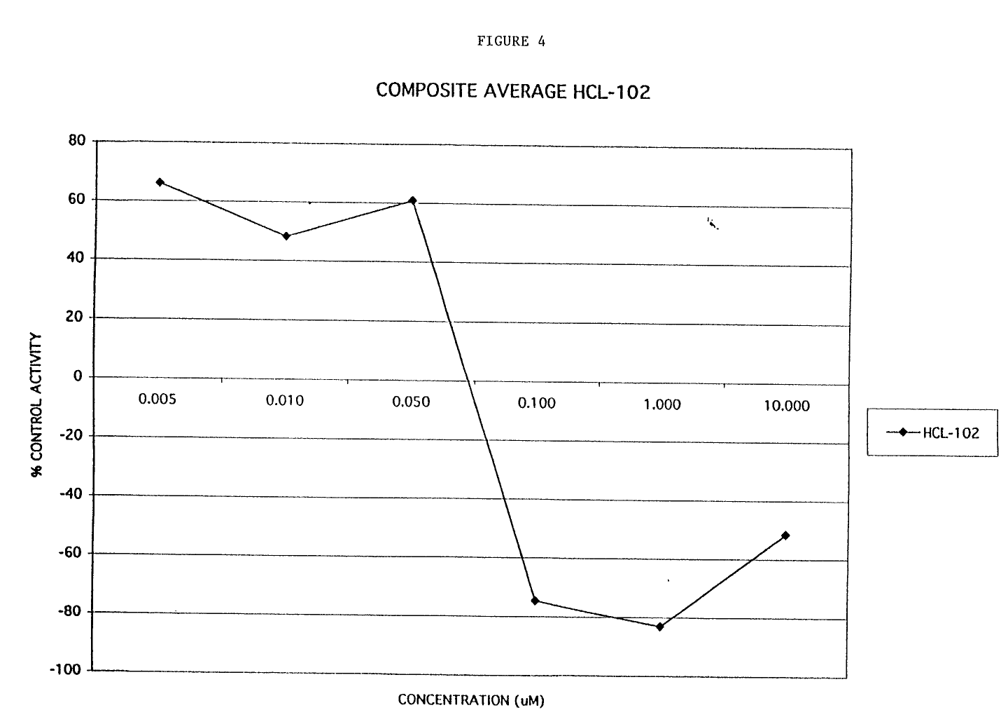

FIG. 5 is a line graph showing the effects of increasing concentrations of antisense oligonucleotide HCL-103 (SEQ ID NO: 3) on IL-15 synthesis in CV-1/EBNA cells.

FIG. 6 is a line graph showing the effects of increasing concentrations of antisense oligonucleotide HCL-104 (SEQ ID NO: 4) on IL-15 synthesis in CV-1/EBNA cells.

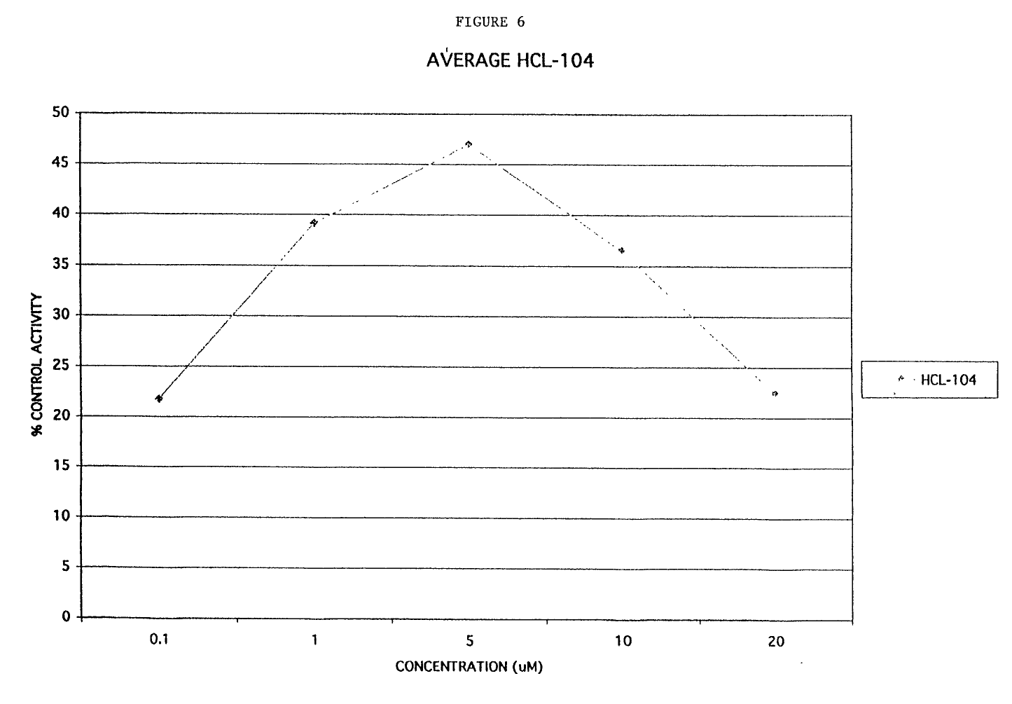

FIG. 7 is a line graph showing the effects of increasing concentrations of antisense oligonucleotide HCL-105 (SEQ ID NO: 5) on IL-15 synthesis in CV-1/EBNA cells.

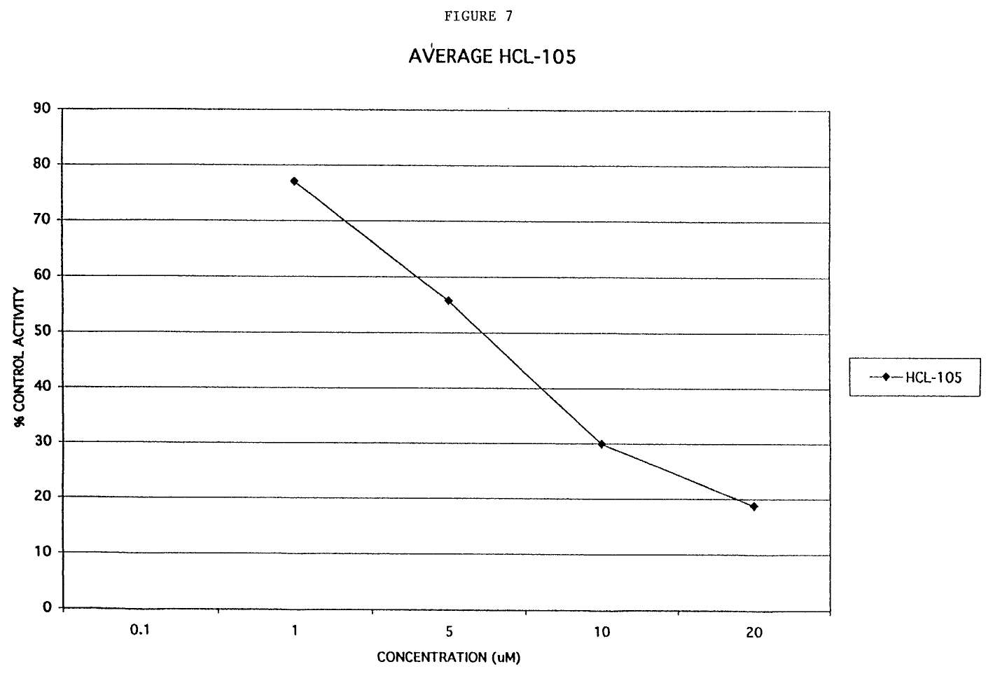

FIG. 8 is a line graph showing the effects of increasing concentrations of antisense oligonucleotide HCL-106 (SEQ ID NO: 6) on IL-15 synthesis in CV-1/EBNA cells.

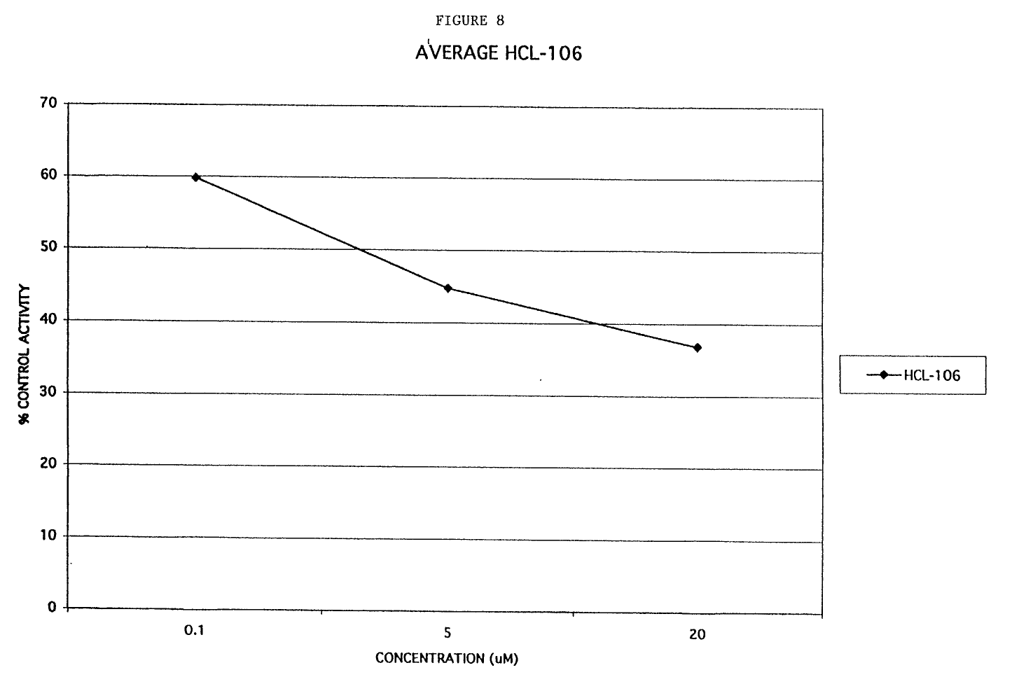

FIG. 9 is a line graph showing the effects of increasing concentrations of antisense oligonucleotide HCL-107 (SEQ ID NO: 7) on IL-15 synthesis in CV-1/EBNA cells.

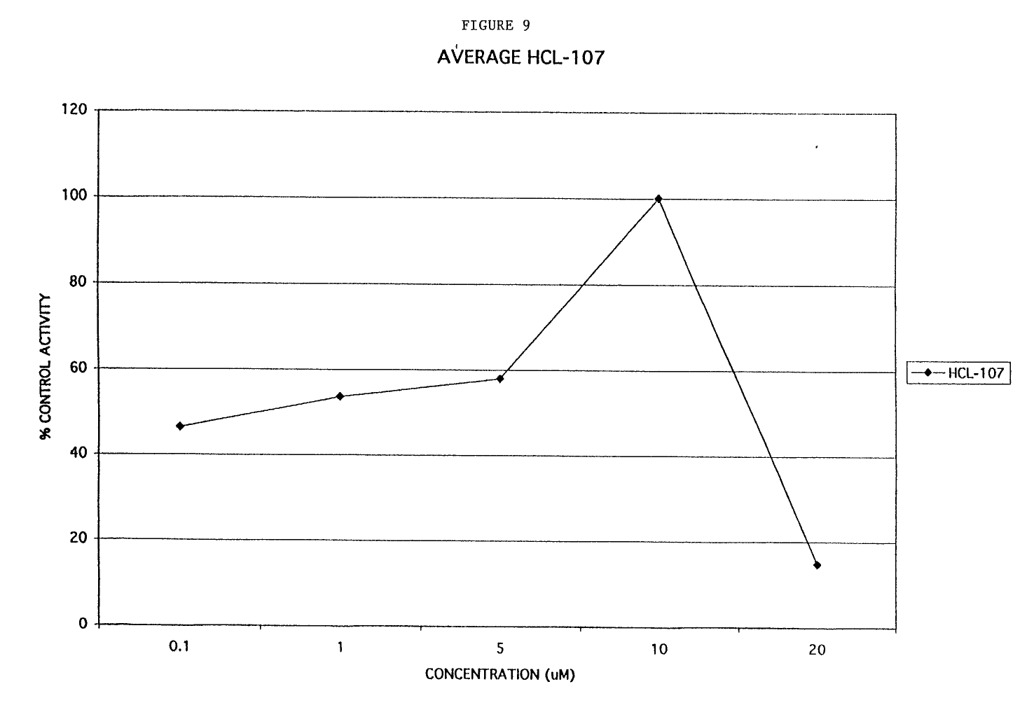

FIG. 10 is a line graph showing the effects of increasing concentrations of antisense oligonucleotide HCL-108 (SEQ ID NO: 8) on IL-15 synthesis in CV-1/EBNA cells.

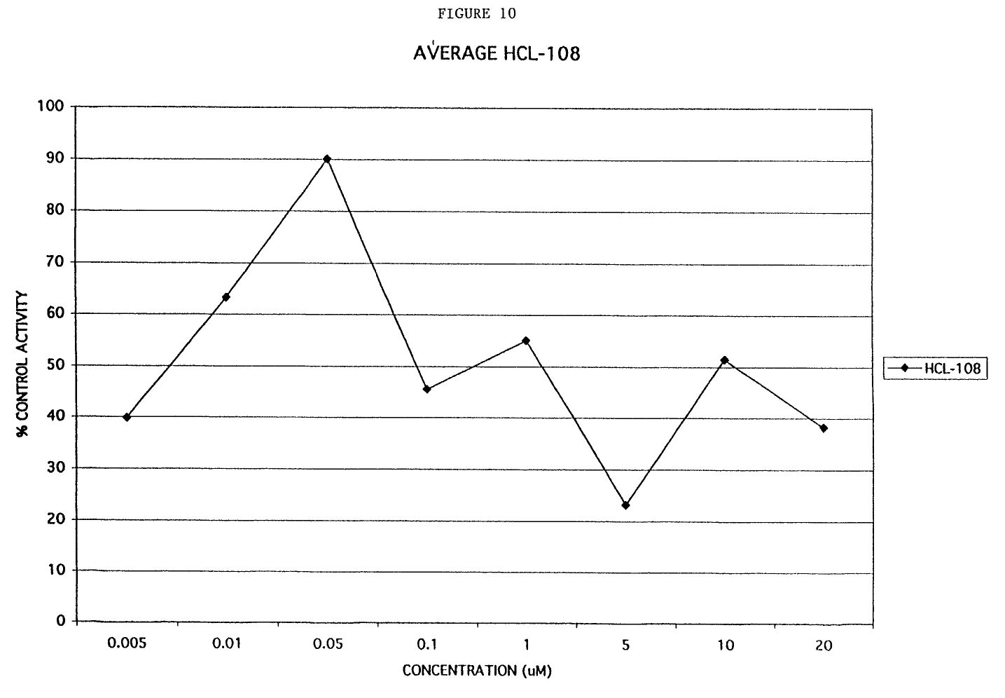

FIG. 11 is a line graph showing the effects of increasing concentrations of antisense oligonucleotide HCL-102 L1 (SEQ ID NO: 10), HCL-102 L2 (SEQ ID NO: 11) and HCL-102 L3 (SEQ ID NO: 12) on IL-15 synthesis in CV-1/EDNA cells.

FIG. 12 is a line graph showing the effects of increasing concentrations of antisense oligonucleotide HCL-102 R1 (SEQ ID NO: 13), HCL-102 L2 (SEQ ID NO: 14) and HCL-102 R3 (SEQ ID NO: 15) on IL-15 synthesis in CV-1/EBNA cells.

FIG. 13 is a line graph showing the effects of increasing concentrations of antisense oligonucleotide SCRAMBLED (SEQ ID NO: 16) on IL-15 synthesis in CV-1/EBNA cells.

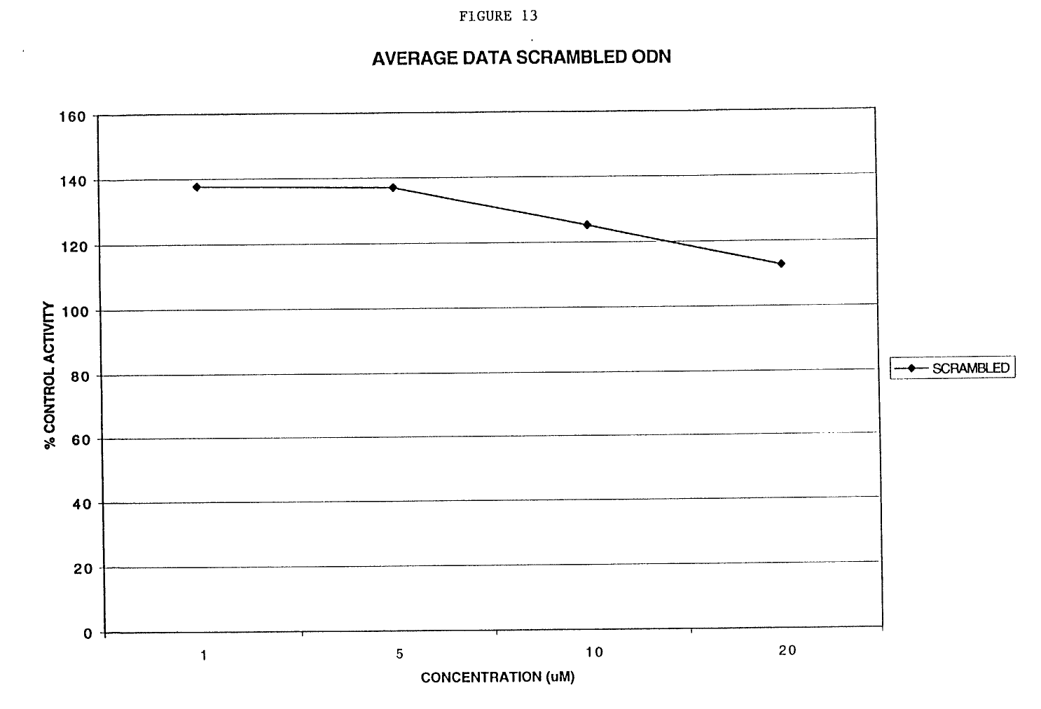

FIG. 14 shows truncated antisense oligonucleotide sequences (SEQ ID NOs: 10-15) designed from the HCL-102 (SEQ ID NO: 2) oligonucleotide. L and R designate left and right truncations from the 5′ and 3′ end, respectively.

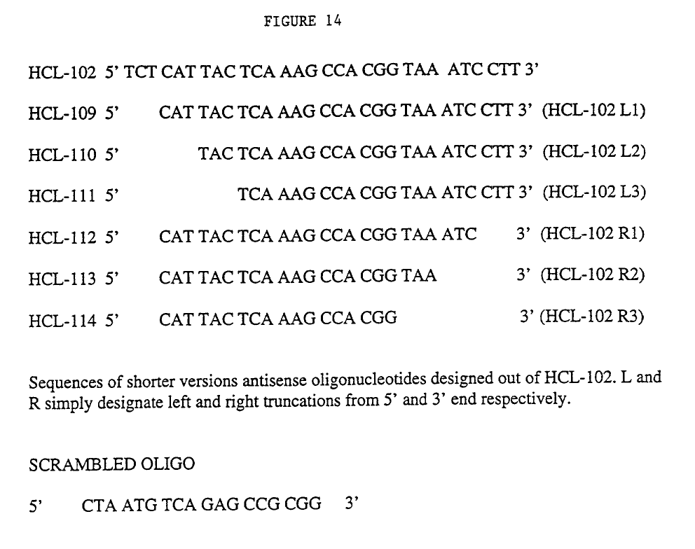

### DETAILED DESCRIPTION

The antisense oligonucleotides of the present invention can effectively reduce IL-15 expression and can be used to treat disease associated with IL-15, such as rheumatoid arthritis (RA), inflammatory bowel disease, cirrhosis, multiple sclerosis chronic liver disease, ulcerative colitis and cell proliferative disorders. The antisense oligonucleotides can be delivered to cells in culture or to cells or tissues in humans or delivered in animal models having these diseases. Binding of IL-15 by an antisense oligonucleotide of the invention can be used to inhibit inflammatory cell function and/or cell recruitment as well as alleviate disease symptoms.

It has been shown that IL-15 is present in RA synovium, inflammatory bowel tissues, ulcerative colitis, cerebral spinal fluid from multiple sclerosis patients, and that IL-15 can induce TNF-α production in RA through the activation of synovial T cells (McInnes et al., 1996, Nature Medicine, 2:175-82; Sakai et al., 1998 Gastroenterology, 114(6):1237-1243; Kivisakk et al., 1998 Clin Exp Immunol, 111(1):193-197; Kacani et al., 1997 Clin Exp Immunol, 108(1):14-18; and Kirman et al., 1996 Am J Gastroenterol, 91(9):1789-1794). Moreover, it was shown that peripheral blood (PB) T cells activated by IL-15 induced significant TNF-α production by macrophages via a cell contact dependent mechanism (McInnes et al., 1997, Nature Medicine 3:189-195). IL-15 has also been shown to be a chemoattractant for T cells in vitro, and can induce proliferation of peripheral blood and synovial cells (McInnes et al., op. cit.), and may play a role in activating antigen specific Th₁ cells. The present invention provides useful antisense oligonucleotides directed against polynucleotides encoding IL-15 to reduce levels of IL-15 protein, thereby affecting the immunologic and chemoattractant functions of this molecule.

“Antisense oligonucleotide” means any RNA or DNA molecules which can bind specifically with a targeted polynucleotide sequence, interrupting the expression of that gene's protein product. The antisense molecule binds to either the messenger RNA forming a double stranded molecule which cannot be translated by the cell or to the DNA or other polynucleotide encoding IL-15. Antisense oligonucleotides of about 8 to 40 nucleic acids and more preferably about 13-30 are preferred since they are easily synthesized and have an inhibitory effect just like antisense RNA molecules. In addition, chemically reactive groups, such as iron-linked ethylenediaminetetraacetic acid (EDTA-Fe) can be attached to an antisense oligonucleotide, causing cleavage of the RNA at the site of hybridization. These and other uses of antisense methods to inhibit the in vitro translation of genes are well known in the art (Marcus-Sakura, 1988, Anal., Biochem., 172:289).

Antisense oligonucleotides are DNA or RNA molecules that are complementary to, at least a portion of, a specific polynucleotide molecule (Weintraub, Scientific American, 262:40, 1990). In the cell, the antisense oligonucleotides hybridize to the corresponding target polynucleotide, forming a double-stranded or triplex molecule. The antisense oligonucleotides interfere with the translation of, for example, mRNA, since the cell will not translate a mRNA that is double-stranded. Antisense oligomers of about 8 to 40 nucleotides are preferred, since they are easily synthesized and are less likely to cause problems than larger molecules when introduced into the target IL-15 producing cell.

Use of a oligonucleotides to stall transcription is known as the triplex strategy since the oligomer winds around double-helical DNA, forming a three-strand helix. Therefore, these triplex compounds can be designed to recognize a unique site on a chosen gene (Maher, et al., 1991, Antisense Res. and Dev., 1(3):227; Helene, C., 1991, Anticancer Drug Design, 6(6):569).

These and other uses of antisense methods to inhibit the in vivo transcription or translation of genes are well known in the art (e.g., De Mesmaeker, et al., 1995. Backbone modifications in oligonucleotides and peptide nucleic acid systems. Curr. Opin. Struct. Biol. 5:343-355; Gewirtz, A. M., et al., 1996b. Facilitating delivery of antisense oligodeoxynucleotides: Helping antisense deliver on its promise; Proc. Natl. Acad. Sci. U.S.A. 93:3161-3163; Stein, C. A. A discussion of G-tetrads 1996. Exploiting the potential of antisense: beyond phosphorothioate oligodeoxynucleotides. Chem. and Biol. 3:319-323).

As used herein, the term “nucleic acid,” “polynucleotide,” “oligonucleotide” or “nucleic acid sequence” refers to a polymer of deoxyribonucleotides or ribonucleotides, in the form of a separate fragment or as a component of a larger construct. For example, nucleic acids can be assembled from cDNA fragments or from polynucleotides to generate a synthetic gene which is capable of being expressed in a recombinant transcriptional unit. Oligonucleotide or nucleic acid sequences of the invention include DNA, RNA, and cDNA sequences.

A “promoter” is a minimal DNA sequence sufficient to direct transcription of a DNA sequence to which it is operably linked. A “promoter” also includes promoter elements sufficient for promoter-dependent gene expression controllable for cell-type specific expression, tissue-specific expression, or inducible by external signals or agents; such elements may be located in the 5′ or 3′ regions of the native gene.

The term “operably associated” refers to functional linkage between the regulatory (e.g. promoter) sequence and the nucleic acid regulated by the regulatory sequence. The operably linked regulatory sequence controls the expression of the product. The regulatory sequence may be heterologous to the desired gene sequence.

A “vector” is any compound or formulation, biological or chemical, that facilitates transformation or transfection of a target cell with a polynucleotide of interest, for example antisense oligonucleotides. Exemplary biological vectors include viruses, particularly attenuated and/or replication-deficient viruses. Exemplary chemical vectors include lipid complexes and DNA constructs.

To “inhibit” or “inhibiting” activity is to reduce that activity a measurable amount, preferably a reduction of at least 30% or more. Where there are multiple different activities that may be inhibited (for example, antisense molecules that bind polynucleotides encoding IL-15 may have the ability to reduce expression of the IL-15 protein, the recruitment of macrophages, and may also have the ability to decrease T cell proliferation), the reduction of any single activity (with or without the other activities) is sufficient to fall within the scope of this definition.

To “specifically bind” is to preferably hybridize to a particular polynucleotide species. The specificity of the hybridization can be modified and determined by standard molecular assays known to those skilled in the art.

A “suppressive-effective” amount is that amount of the construct, and thus antisense, administered in an amount sufficient to suppress the expression of the target, e.g., inhibit translation of mRNA, by at least 75% of the normal expression, and preferably by at least 90%. The effectiveness of the construct can be determined phenotypically or by standard Northern blot analysis or immunohistochemically, for example. Other standard nucleic acid detection techniques or alternatively immunodiagnostic techniques will be known to those of skill in the art (e.g., Western or Northwestern blot analysis).

A “transgenic animal” is an animal that includes a transgene that is inserted into an embryonal cell and becomes a part of the genome of the animal which develops from that cell, or an offspring of such an animal. In the transgenic animals described herein, the transgene causes specific tissue cells to express an antisense oligonucleotide which specifically binds IL-15 polynucleotide. Any animal that can be produced by transgenic technology is included in the invention, although mammals are preferred. Preferred mammals include non-human primates, sheep, goats, horses, cattle, pigs, rabbits, and rodents such as guinea pigs, hamsters, rats, gerbils, and mice.

A “transgene” is a DNA sequence that includes one or more selected DNAs, e.g., encoding antisense oligonucleotides that bind mRNA encoding IL-15, to be expressed in a transgenic animal, which is partly or entirely heterologous, i.e., foreign, to the transgenic animal, or homologous to an endogenous gene of the transgenic animal, but which is designed to be inserted into the animal's genome at a location which differs from that of the natural gene. A transgene includes one or more promoters and any other DNA, such as introns, necessary for expression of the selected DNA, all operably linked to the selected DNA, and may include an enhancer sequence.

A “disorder associated with IL-15” or “disease associated with IL-15” is any disease state associated with the expression of IL-15. An example of such disorders include rheumatoid arthritis, inflammatory bowel disease, cirrhosis, multiple sclerosis, chronic liver disease, ulcerative colitis and cell proliferative disorders.

Antisense Oligonucleotides

The present invention provides a method for ameliorating or inhibiting the production of IL-15 in diseases associated with IL-15 production. Inhibition of IL-15 production is achieved by administering to the cell, tissue or subject an antisense oligonucleotide sequence which is capable of hybridizing to the nucleic acid sequence of an IL-15 polynucleotide. This antisense oligonucleotide inhibits, or down regulates the expression of the IL-15 gene product in the cell, tissue or subject.

The invention additionally provides antisense oligonucleotides which reduce expression of IL-15. An antisense oligonucleotide of the invention has a sequence that is complementary to, and thus hybridizes with the nucleic acid sequence of the target IL-15 polynucleotide. However, absolute complementarity is not required. The polynucleotide sequence of the target IL-15 sequence can be either a DNA or an RNA sequence. The target includes sequence upstream from the 5′ terminus of the structural gene, such as regulatory sequences, and sequences downstream from the 3′ terminus of the structural gene. An antisense oligonucleotide is “complementary” to the target IL-15 oligonucleotide, and thus useful according the invention, if it is capable of forming a stable duplex or triplex with, at least part of, the target polynucleotide sequence of the target so that processing, transcription or translation of the polynucleotides is inhibited, or capable of forming a complex, such as a triplex, with genomic DNA of the gene so that promotion of transcription is inhibited or premature transcript termination is produced. (Green et al., 1990 Clinical Biotechnology, 2:75). When the antisense molecule hybridizes to the target polynucleotide, stable duplex or triplex formation depends on the sequence and length of the hybridizing polynucleotide and the degree of complementarity between the antisense molecule and the target sequence. The system can tolerate less fidelity (complementarity) when a longer oligonucleotide is used. However, oligonucleotides of about 8 to about 40 bases in length and having sufficient complementarily to form a duplex having a melting temperature of greater than about 40° C. under physiological conditions are particularly well suited for practice of the invention (Thoung, et al., 1987 PNAS USA, 84:5129; Wilson et al., 1988 Nucleic Acids Res., 16:5137; Maniatis, et al., Molecular Cloning: A Laboratory Manual, Old Spring Harbor Laboratory Press, Cold Spring Harbor, N.Y., 1982). Accordingly, such oligonucleotides are preferred.

The antisense molecules of the invention have a specific substrate binding portion which is complementary to a target region of IL-15 polynucleotides, and have nucleotide sequences within or surrounding the substrate binding site which impart the ability to selectively hybridize to relative portions of the IL-15 polynucleotide. Eight illustrated target binding sequences, corresponding to antisense molecules having SEQ ID NOs: 1-8 and 10-15 are provided and described herein. These exemplary antisense molecules were designed to hybridize to various sites on the IL-15 mRNA (SEQ ID NO: 9).

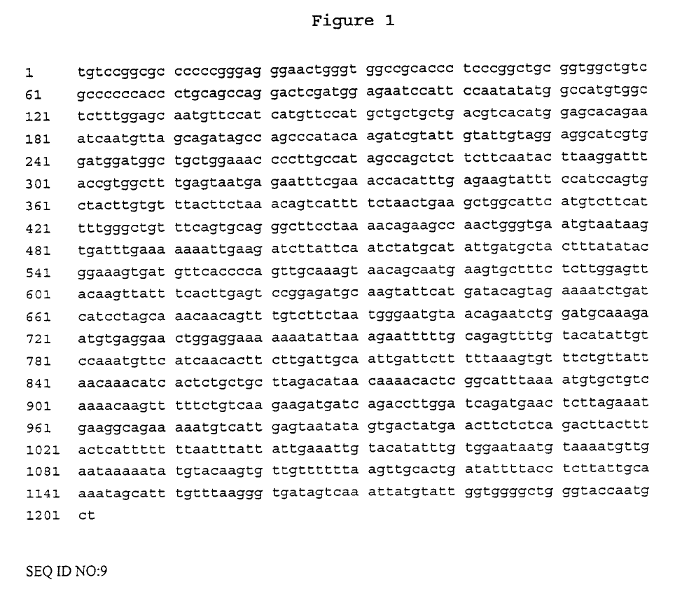

The exemplary antisense molecules of the invention are targeted for binding to the IL-15 sequence (SEQ ID NO: 9), for example, SEQ ID NO: 1 binds at positions 306-323; SEQ ID NO: 2 binds at positions 293-322; SEQ ID NO: 3 binds at positions 314-343; SEQ ID NO: 4 binds at positions 358-378; SEQ ID NO: 5 binds at positions 440-466; SEQ ID NO: 6 binds at positions 545-569; SEQ ID NO: 7 binds at positions 673-697; SEQ ID NO: 8 binds at positions 712-735; SEQ ID NO: 10 binds at positions 293-319; SEQ ID NO: 11 binds at positions 293-316; SEQ ID NO: 12 binds at positions 293-313; SEQ ID NO: 13 binds at positions 296-319; SEQ ID NO: 14 binds at positions 299-319; and SEQ ID NO: 15 binds at positions 302-319. Binding positions are from the ATG of the open reading frame (position 317 for human IL-15 GenBank U14407 and position 1 for the purposes of the invention; see FIG. 1).

The invention includes antisense oligonucleotides which hybridize with a polynucleotide sequence comprising SEQ ID NO: 9 or its complement. The antisense oligonucleotides employed may be unmodified or modified RNA or DNA molecules. Suitable modifications include, but are not limited to, the ethyl or methyl phosphorate modification disclosed in U.S. Pat. No. 4,469,863, the disclosure of which is incorporated by reference, and the phosphorthioate modifications to deoxynucleotides described by LaPlanche, et at., 1986 Nucleic Acids Research, 14:9081, and by Stec, et al., 1984 J. Am. Chem Soc. 106:6077. The modification to the antisense oligonucleotides is preferably a terminal modification in the 5′ or 3′ region. Preferred are modifications of the 3′ terminal region. Also preferred are modifications with methyl groups added to 5′ carbon atoms as described by Saha, et al., 1993 CEN, 44:44.

Phosphodiester-linked oligonucleotides are particularly susceptible to the action of nucleases in serum or inside cells, and therefore in a preferred embodiment the antisense molecules of the present invention are phosphorothioate or methyl phosphonate-linked analogues, which have been shown to be nuclease-resistant. Specific examples of some preferred oligonucleotides envisioned for this invention may contain phosphorothioates, phosphotriesters, methyl phosphonates, short chain alkyl or cycloalkyl intersugar linkages or short chain heteroatomic or heterocyclic intersugar (“backbone”) linkages. Most preferred are phosphorothioates and those with CH₂NHOCH₂, CH₂N(CH₃)OCH₂, CH₂ON(CH₃) CH₂, CH₂N(CH₃)N(CH₃)CH₂ and ON(CH₃)CH₂CH₂ backbones (where phosphodiester is OPOCH₂). Also preferred are oligonucleotides having morpholino backbone structures (Summerton, J. E. and Weller, D. D., U.S. Pat. No. 5,034,506). In other preferred embodiments, 2′-methylribonucleotides (Inoue, et al., 1987 Nucleic Acids Research, 15:6131) and chimeric oligonucleotides that are composite RNA-DNA analogues (Inoue, et al., 1987 FEBS Lett., 215:327) may also be used for the purposes described herein. Finally, DNA analogues, such as peptide nucleic acids (PNA) are also included (Egholm, et al., 1993 Nature 365:566; P. E. Nielsen, M. Egholm, R. H. Berg, O. Buchardt, 1991 Science, 254:1497) and can be used according to the invention. Other preferred oligonucleotides may contain alkyl and halogen-substituted sugar moieties comprising one of the following at the 2′ position: OH, SH, SCH₃, F, OCN, OCH₃OCH₃, OCH₃O(CH₂)nCH₃, O(CH₂)nNH₂ or O(CH₂)nCH3 where n is from 1 to about 10; C1 to C10 lower alkyl, substituted lower alkyl, alkaryl or aralkyl; C1; Br; CN; CF₃; OCF₃; O, S , or N-alkyl; O, S or N alkenyl; SOCH₃; OS₂CH₃; ONO₂; NO₂; N₃; NH₂; heterocycloalkyl; heterocycloalkaryl; aminoalkylamino; polyalkylamino; substituted silyl; an RNA cleaving group; a cholesteryl group; a conjugate; a reporter group; an intercalator; a group for improving the pharmacokinetic properties of a oligonucleotide; or a group for improving the pharmacodynamic properties of a oligonucleotide and other substituents having similar properties. Oligonucleotides may also have sugar mimetics such as cyclobutyls in place of the pentofuranosyl group. Other preferred embodiments may include at least one modified base form or “universal base” such as inosine. The preparation of base-modified nucleosides, and the synthesis of modified oligonucleotides using said base-modified nucleosides as precursors, has been described, for example, in U.S. Pat. Nos. 4,948,882 and 5,093,232. These base-modified nucleosides have been designed so that they can be incorporated by chemical synthesis into either terminal or internal positions of a oligonucleotide. Such base-modified nucleosides, present at either terminal or internal positions of a oligonucleotide, can serve as sites for attachment of a peptide or other antigen. Nucleosides modified in their sugar moiety have also been described (e.g., U.S. Pat. No. 5,118,802) and can be used similarly. Persons of ordinary skill in this art will be able to select other linkages for use in the invention. These modifications also may be designed to improve the cellular uptake and stability of the oligonucleotides. It is understood that depending on the route or form of administration of the antisense oligonucleotides of the invention, the modification or site of modification will vary (e.g., 5′ or 3′ modification). One of skill in the art could readily determine the appropriate modification without undue experimentation.

In order for the target cell, tissue or subject to be rendered susceptible to the antisense oligonucleotides in accordance with the method of the invention, the cells must be exposed to the oligonucleotide under condition that facilitate their uptake by the cell, tissue or subject. In vitro therapy may be accomplished by a number of procedures, including, for example, simple incubation of the cells or tissue with the oligonucleotide in a suitable nutrient medium for a period of time suitable to inhibit IL-15 production.

The antisense oligonucleotides of the invention can be delivered alone or in conjunction with other agents such as immunosuppressive drugs, ribozymes or other antisense molecules. For example, ribozymes or antisense molecules that specifically bind mRNA encoding another cytokine, such as TNF-α or interferon-γ, can be used with the antisense molecules of the present invention. Further, combinations of the antisense molecules of the invention, e.g., SEQ ID NO: 1-8 and 10-15, can be used. Agents useful n treating rheumatoid arthritis, such as colloidal gold or methotrexate, may also be used in conjunction with the antisense molecules which specifically bind IL-15. Anti-inflammatory agents, such as non-steroidal anti-inflammatory drugs, corticosteroids, and hydroxychloroquine, immunosuppressive agents such as cyclosporine, and cytotoxic drugs such as cyclophosphamide, azathioprine, may also be used in conjunction with the antisense molecules of the invention.

Additionally, the antisense oligonucleotides of the present invention may be administered ex vivo by harvesting cells or tissue from a subject, treating them with the antisense oligonucleotide, then returning the treated cells or tissue to the subject. The present invention provides method for the treatment of a disease which is associated with IL-15. Such therapy would achieve its therapeutic effect by introduction of the appropriate antisense oligonucleotide which binds polynucleotides encoding IL-15 into cells of subjects having the disorder. Delivery of the IL-15 antisense molecule can be achieved using a recombinant expression vector such as a chimeric virus or a colloidal dispersion system.

Many of the methods as described herein can be performed in vivo or ex vivo. Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). Preferably, when the subject is a human, a vector such as the gibbon ape leukemia virus (GaLV) is utilized. A number of additional retroviral vectors can incorporate multiple genes. All of these vectors can transfer or incorporate a gene for a selectable marker so that transduced cells can be identified and generated. By inserting a sequence encoding an antisense oligonucleotide which specifically binds polynucleotides encoding IL-15 into the viral vector, along with another gene which encodes the ligand for a receptor on a specific target cell, for example, the vector is now target specific. Preferred targeting is accomplished by using an antibody to target the retroviral vector. Those of skill in the art will know of, or can readily ascertain without undue experimentation, specific polynucleotide sequences which can be inserted into the retroviral genome, for example, to allow target specific delivery of the retroviral vector containing the antisense oligonucleotide.

Since recombinant retroviruses are defective, they require assistance in order to produce infectious vector particles. This assistance can be provided, for example, by using helper cell lines that contain plasmids encoding all of the structural genes of the retrovirus under the control of regulatory sequences within the LTR. These plasmids are missing a nucleotide sequence which enables the packaging mechanism to recognize an RNA transcript for encapsidation. Helper cell lines which have deletions of the packaging signal include but are not limited to ψ2, PA317 and PA12, for example. These cell lines produce empty virions, since no genome is packaged. If a retroviral vector is introduced into such cells in which the packaging signal is intact, but the structural genes are replaced by other genes of interest, the vector can be packaged and vector virion produced.

Alternatively, NIH 3T3 or other tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

Another targeted delivery system for antisense oligonucleotides that bind polynucleotides encoding IL-15 is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. It has been shown that large unilamellar vesicles (LUV), which range in size from 0.2-4.0 μm can encapsulate a substantial percentage of an aqueous buffer containing large macromolecules. RNA, DNA and intact virions can be encapsulated within the aqueous interior and be delivered to cells in a biologically active form (Fraley, et al., 1981 Trends Biochem. Sci., 6:77). In order for a liposome to be an efficient gene transfer vehicle, the following characteristics should be present: (1) encapsulation of the genes of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., 1988 Biotechniques, 6:682).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidyletha-nolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidyl-choline.

The targeting of liposomes has been classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. In general, the compounds bound to the surface of the targeted delivery system will be ligands and receptors which will allow the targeted delivery system to find and “home in” on the desired cells. A ligand may be any compound of interest which will bind to another compound, such as a receptor.

Another delivery system for the antisense oligonucleotides of the invention at particular sites in a subject, for instance at a joint site afflicted with rheumatoid arthritis, includes the use of gene-activated matrices. In this system the antisense molecule is coated on a biocompatible matrix, sponge or scaffold and implanted at the tissue site wherein cells proliferate and grow on the scaffold, taking up the antisense oligonucleotide (See for example U.S. Pat. No. 5,763,416, which is incorporated herein by reference).

In yet another delivery system, the antisense molecules of the invention may be microinjected into cells. The antisense molecules may be prepared in an appropriate buffer and the naked oligonucleotide, either alone or contained in an appropriate vector, microinjected, for example, into a stem cell of a tissue to be treated.

In addition, antisense oligonucleotides according to the invention may also be administered in vivo. Antisense oligonucleotides can be administered as the compound or as a pharmaceutically acceptable salt of the compound, alone or in combination with pharmaceutically acceptable carriers, diluents, simple buffers, and vehicles. For example expression vectors that produce antisense molecules can be engineered from DNA duplexes in the laboratory and introduced into cells (Weintraub, et al., 1990 Sci. Amer. 1:40). Most preferably, antisense oligonucleotides are mixed individually or in combination with pharmaceutically acceptable carriers to form compositions which allow for easy dosage preparation.

An antisense oligonucleotide of the invention can be administered to provide in vivo therapy to a subject having a disorder which is associate with IL-15 expression. Such therapy can be accomplished by administering ex vivo and in vivo as the case may be, a therapeutically effective amount of antisense oligonucleotide. The term “therapeutically effective” means that the amount of antisense oligonucleotide administered is of sufficient quantity to suppress, to some beneficial degree, expression of IL-15.

Antisense oligonucleotide according to the present invention can be administered to the patient in any acceptable manner including orally, by injection, using an implant, nasally and the like. Oral administration includes administering an oligonucleotide of the present invention in tablets, suspension, implants, solutions, emulsions, capsules, powders, syrups, water composition, and the like. Nasal administration includes administering the composition of the present invention in sprays, solutions and the like. Injections and implants are preferred because they permit precise control of the timing and dosage levels useful for administration, with injections being most preferred. Antisense oligonucleotides are preferably administered parenterally.

The therapeutic agents useful in the method of the invention can be administered parenterally by injection or by gradual perfusion over time. Administration may be intravenously, intra-peritoneally, intramuscularly, subcutaneously, intra-cavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents and inert gases and the like.

The invention also includes a composition for therapy comprising an effective amount of an enzymatic RNA of the invention or combination thereof, and a physiologically acceptable excipient or carrier.

Physiologically acceptable and pharmaceutically acceptable excipients and carriers are well known to those of skill in the art. By “physiologically or pharmaceutically acceptable carrier” as used herein is meant any substantially non-toxic carrier for administration in which an antisense oligonucleotide of the invention will remain stable and bioavailable when used. For example, the antisense oligonucleotide of the invention can be dissolved in a liquid, dispersed or emulsified in a medium in a conventional manner to form a liquid preparation or is mixed with a semi-solid (gel) or solid carrier to form a paste, ointment, cream, lotion or the like.

Suitable carriers include water, petroleum jelly (vaseline), petrolatum, mineral oil, vegetable oil, animal oil, organic and inorganic waxes, such as microcrystalline, paraffin and ozocerite wax, natural polymers, such as xanthanes, gelatin, cellulose, or gum arabic, synthetic polymers, such as discussed below, alcohols, polyols, water and the like. Preferably, because of its non-toxic properties, the carrier is a water miscible carrier composition that is substantially miscible in water. Such water miscible carrier composition can include those made with one or more ingredients set forth above but can also include sustained or delayed release carrier, including water containing, water dispersable or water soluble compositions, such as liposomes, microsponges, microspheres or microcapsules, aqueous base ointments, water-in-oil or oil-in-water emulsions or gels.

The carrier can comprise a sustained release or delayed release carrier. The carrier is any material capable of sustained or delayed release of the antisense molecule specifically directed against IL-15 polynucleotide to provide a more efficient administration resulting in one or more of less frequent and/or decreased dosage of the antisense molecule, ease of handling, and extended or delayed effects. The carrier is capable of releasing the oligomer when exposed to the environment of the area for diagnosis or treatment or by diffusing or by release dependent on the degree of loading of the oligonucleotide to the carrier in order to obtain release of the antisense oligonucleotide of the invention. Non-limiting examples of such carriers include liposomes, microsponges, microspheres, gene-activated matrices, as described above, or microcapsules of natural and synthetic polymers and the like. Examples of suitable carriers for sustained or delayed release in a moist environment include gelatin, gum arabic, xanthane polymers; by degree of loading include lignin polymers and the like; by oily, fatty or waxy environment include thermoplastic or flexible thermoset resin or elastomer including thermoplastic resins such as polyvinyl halides, polyvinyl esters, polyvinylidene halides and halogenated polyolefins, elastomers such as brasiliensis, polydienes, and halogenated natural and synthetic rubbers, and flexible thermoset resins such as polyurethanes, epoxy resins and the like.

Preferably, the sustained or delayed release carrier is a liposome, microsponge, microsphere or gel.

The compositions of the invention are administered by any suitable means, including injection, implantation, transdermal, intraocular, transmucosal, bucal, intrapulmonary, and oral.

Preferably the carrier is a pH balanced buffered aqueous solution for injection. However, the preferred carrier will vary with the mode of administration.

The compositions for administration usually contain from about 0.0001% to about 90% by weight of the antisense oligonucleotide of the invention compared to the total weight of the composition, preferably from about 0.5% to about 20% by weight of the antisense oligonucleotide of the invention compared to the total composition, and especially from about 2% to about 20% by weight of the antisense oligonucleotide of the invention compared to the total composition.

The effective amount of the antisense oligonucleotide of the invention used for therapy or diagnosis of course can vary depending on one or more of factors such as the age and weight of the patient, the type of formulation and carrier ingredients, frequency of use, the type of therapy or diagnosis preformed and the like. It is a simple matter for those of skill in the art to determine the precise amounts to use taking into consideration these factors and the present specification.

Transgenic Animals

In a further embodiment, a transgenic animal can be developed using a construct containing the antisense oligonucleotide and method of the invention in order to identify the impact of increased or decreased IL-15 levels on a particular pathway or phenotype. The construct can be any number of vectors containing the antisense oligonucleotide of the invention. Protocols useful in producing such transgenic animals are described below. The protocol generally follows conventional techniques for introduction of expressible transgenes into mammals. Those of ordinary skill in the art will be familiar with these applications and will be able to apply the techniques in the context of the present invention without undue experimentation.

For example, embryonic target cells at various developmental stages can be used to introduce transgenes encoding an IL-15 antisense molecule. Different methods are used depending on the stage of development of the embryonic target cell. The zygote is the best target for microinjection. In the mouse, the male pronucleus reaches the size of approximately 20 micrometers in diameter which allows reproducible injection of 1-2 pl of DNA solution. The use of zygotes as a target for gene transfer has a major advantage in that in most cases the injected DNA will be incorporated into the host gene before the first cleavage (Brinster, et al., 1985, Proc. Natl. Acad. Sci. USA 82:4438-4442). As a consequence, all cells of the transgenic non-human animal will carry the incorporated transgene. In general, this will also be reflected in the efficient transmission of the transgene to offspring of the founder since 50% of the germ cells will harbor the transgene. Microinjection of zygotes is a suitable method for incorporating transgenes in practicing the invention.

Retroviral infection can also be used to introduce a transgene encoding an antisense oligonucleotide which specifically binds IL-15 polynucleotides into a non-human animal. The developing non-human embryo can be cultured in vitro to the blastocyst stage. During this time, the blastomeres can be targets for retroviral infection (Jaenisch, 1976, Proc. Natl. Acad. Sci. USA 73:1260-1264). Efficient infection of the blastomeres is obtained by enzymatic treatment to remove the zona pellucida (Hogan, et al., 1986, Manipulating the Mouse Embryo, Cold Spring Harbor Laboratory Press, Cold Spring Harbor, N.Y.). The viral vector system used to introduce the transgene encoding an antisense oligonucleotide which specifically binds IL-15 polynucleotides is typically a replication-defective retrovirus carrying the transgene (Jahner, et al., 1985, Proc. Natl. Acad. Sci. USA 82:6927-6931; Van der Putten, et al., Proc Natl. Acad. Sci. USA 82:6148-6152). Transfection is easily and efficiently obtained by culturing the blastomeres on a monolayer of virus-producing cells (Van der Putten, supra; Steward, et al., 1987, EMBO J., 6:383-388).

Alternatively, infection can be performed at a later stage. Virus or virus-producing cells can be injected into the blastocoele (Jahner, et al., 1982, Nature, 298:623-628). Most of the founder animals will be mosaic for the transgene since incorporation occurs only in a subset of the cells which formed the transgenic non-human animal. Further, the founder animals may contain various retroviral insertions of the transgene at different positions in the genome which generally will segregate in the offspring. In addition, it is also possible to introduce transgenes encoding an IL-15 antisense oligonucleotide into the germ line, albeit with low efficiency, by intrauterine retroviral infection of the midgestation embryo (Jahner, et al., supra, 1982).

A third type of target cell for introduction of heterologous nucleic acid sequences is the embryonal stem cell (ES). ES cells are obtained from pre-implantation embryos cultured in vitro and fused with embryos (Evans, et al., 1981, Nature, 292:154-156; Bradley, et al., 1984, Nature, 309:255-258,; Gossler, et al., 1986, Proc. Natl. Acad. Sci. USA, 83:9065-9069; and Robertson, et al., 1986, Nature, 322:445-448). Transgenes encoding antisense oligonucleotides which specifically bind IL-15 polynucleotides can be efficiently introduced into the ES cells by DNA transfection or by retro-virus-mediated transduction. These transformed ES cells can thereafter be combined with blastocysts from a non-human animal. The ES cells will thereafter colonize the embryo and contribute to the germ line of the resulting chimeric animal (see for review, Jaenisch, 1988, Science, 240:1468-1474). Any ES cell may be used is accordance with the present invention. It is, however, preferred to use primary isolates of ES cells. Such isolates may be obtained directly from embryos such as with the CCE cell line disclosed by Robertson, E. J., in Current Communications in Molecular Biology, Capecchi, M. R. (Ed.) Cold Springs Harbor Press, Cold Springs Harbor, N.Y. (1989), pp.39-44, or from the clonal isolation of ES cells from the CCE cell line (Scwartzberg, P. A. et al., 1989, Science 246:799). ES cells may be derived or isolated from any species, although cells derived or isolated from mammals such as rodents, rabbits, and non-human primates are preferred.

The cre/lox system as described in U.S. Pat. No. 4,959,317, incorporated herein by reference, can be utilized in the production of transgenic animals. A first and second loxP DNA sequence is introduced into cells connected by a preselected antisense or replacement gene, such as an antisense oligonucleotide which specifically binds IL-15 polynucleotides, herein referred to as a “target transgene”. The “target transgene” of interest can be a complete gene or any other sequence of nucleotides including those of homologous, heterologous, or synthetic origin. The target transgene sequence can be for example, an antisense or replacement gene for a structural protein, an enzyme, a regulatory molecule, or a cytokine such as IL-15. The target transgene may also be a gene of undetermined function. Using tissue-specific or developmentally-specific regulatory sequences (as described above) to direct expression of the target transgene, a function could be identified. If the first and second lox sites have the same orientation (direct repeats), activation of the regulatory nucleotide sequence of the transactivator transgene results in a deletion of the target transgene DNA, such that ablation or modification of activity results. If the first and second lox sites have opposite orientation (inverted repeats), activation of the regulatory nucleotide produces an inversion of the nucleotide sequence of the target transgene.

The construct of the invention may be used to introduce DNA sequences into the germ line cells of “non-humans” to create transgenic animals. Mice are useful as transgenic animals. However, other non-humans of the invention include but are not limited to other rodents (e.g., rat, hamster), rabbits, chickens, sheep, goats, pigs, cattle, and non-human primates.

Materials and Methods

Antisense oligonucleotides targeting IL-15 mRNA (HCL-101-114 (SEQ ID NOs: 1-8 and 10-15)) were synthesized as phosphorothioate derivatives. Purified oligonucleotides (ODN) were tested for purity and homogeneity by end-labeling of DNA using T4 polynucleotide kinase. These purified ODN were tested for inhibition of IL-15 synthesis by CV-1/EBNA cells, if any. Cells plated at a density of 20,000 cells/well were transfected in a 96-well culture dish. Cells were washed two times with serum free pre-warmed medium (DMEM). DMEM containing 2 μg/ml of lipofectin (BRL) was added to each well of the plate (100 μl). This pretreatment of cells with lipofectin (BRL) was added to each well of the plate in order to enhance ODN uptake. ODN were then added as a 20×stock solution to the wells and incubated for 5 h at 37° C. Medium was removed and replaced with the 5% FBS containing medium (150 μl) with various concentrations of ODN. Cells were incubated for an additional 3-4 h at 37° C. and then stimulated with IFN-γ (100 units/ml) for 15-18 h. IL-15 expression was tested in culture supernatants utilizing ELISA (Quantikine human IL-15 ELISA kit from R & D Systems). The optical density (OD) received as a result of the color reaction was converted to the concentration of IL-15 (pg/ml) produced by the cells. The data were expressed as percent control activity which was calculated as follows: {[IL-15 expression for oligonucleotide-treated IFN-induced cells)−(basal IL-15 expression)]/[(IFN-induced IL-15 expression)−(basal IL-15 expression]}×100. Both basal and untreated cells were pretreated with lipofectin. The accuracy of each individual experiment was cross-checked with IL-15 production in normal cells (with no lipofectin treatment).

Results

Phosphorothioate antisense oligonucleotides targeting eight different sites on IL-15 mRNA were tested for inhibition of IL-15 synthesis in CV-1/EBNA cells. These cells were chosen because their IL-15 expression level was higher than several other cell lines tested (A549, PMC2, SW982). Further, IL-15 expression was inducible by culturing the cells in the presence of IFN-γ (100 units/ml) in a concentration-dependent manner at 37° C. in 5% CO₂ for up to 18 h. IL-15 expression was determined by ELISA using the cell culture supernatant.

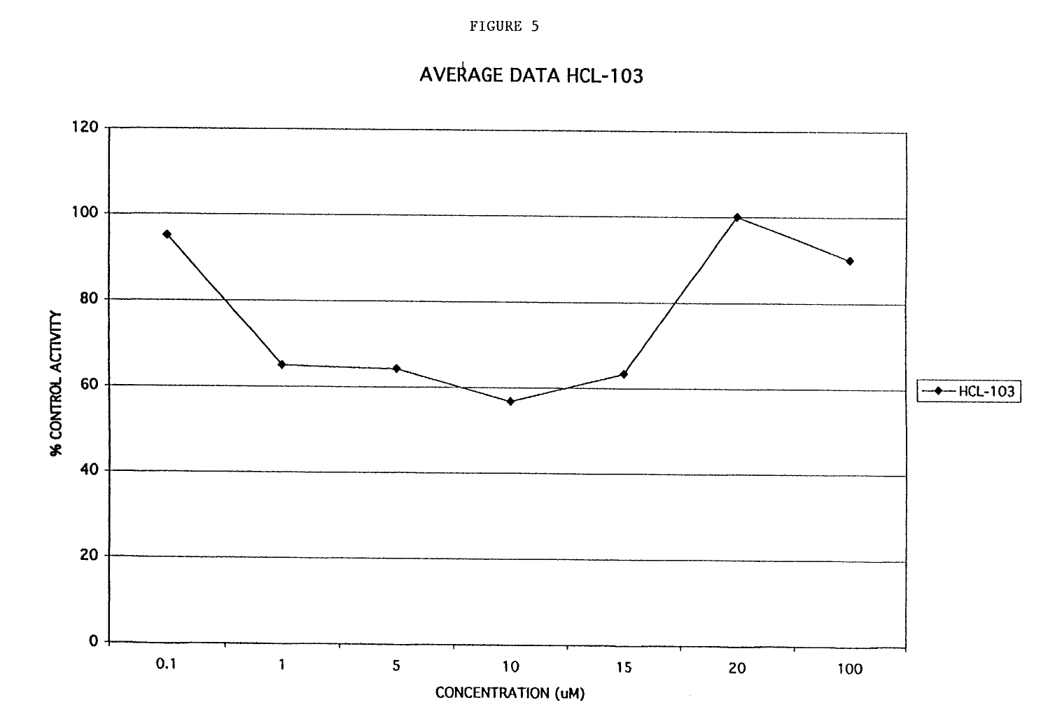

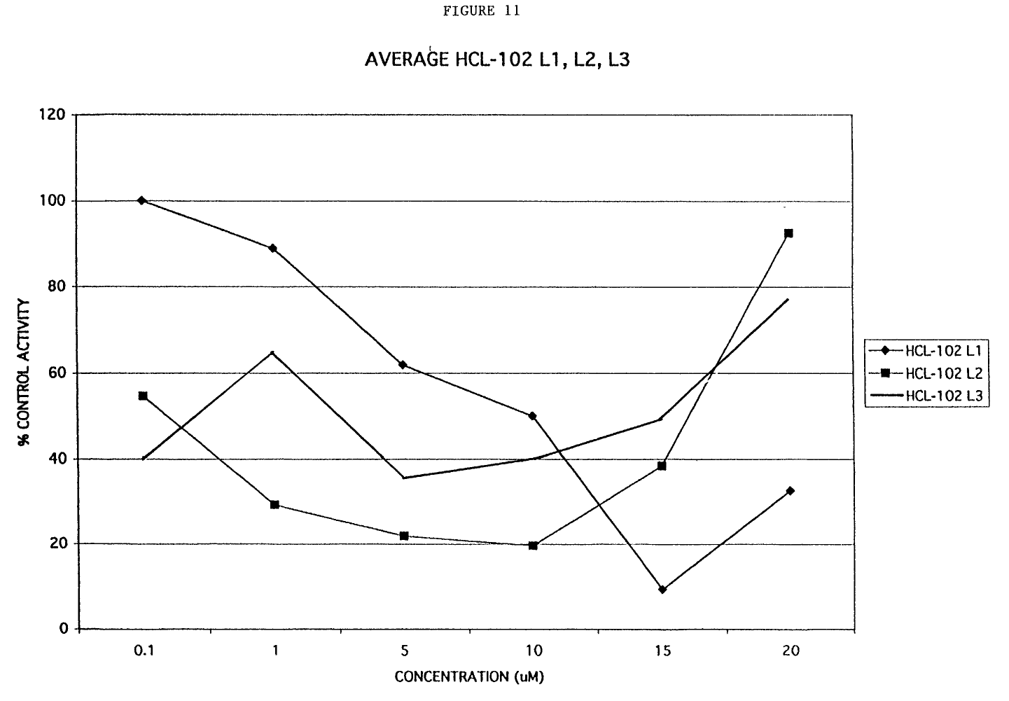

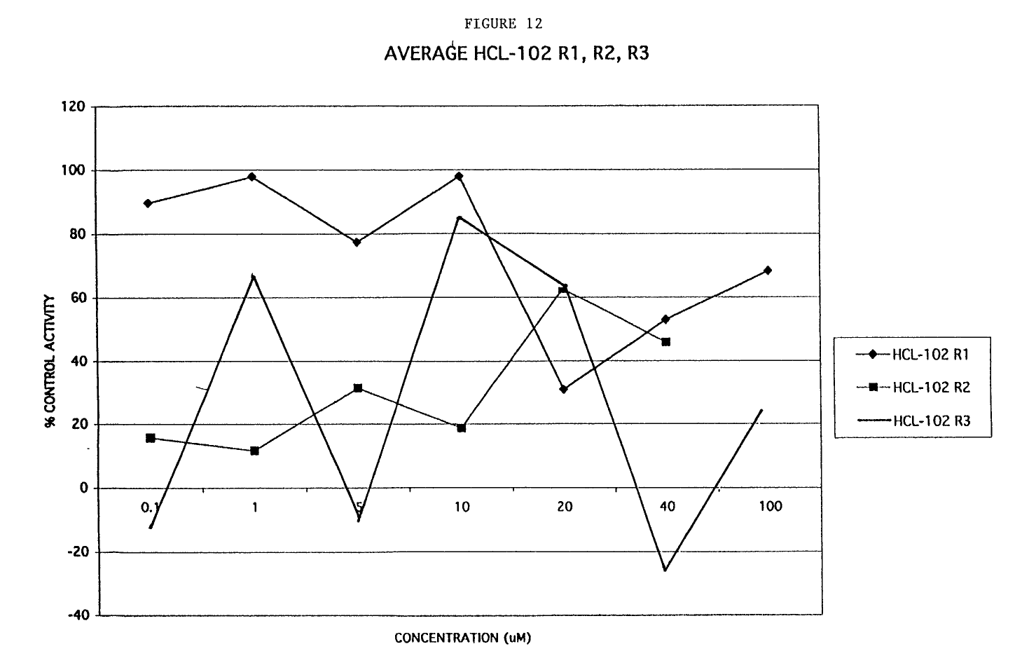

Of the eight oligonucleotides tested (HCL-101 through 108 (SEQ ID NOs: 1-8)), HCL-102 (SEQ ID NO: 2) inhibited IL-15 production to the greatest extent. The data represents the average activity for SEQ ID NOs: 1-8 from three separate experiments. HCL-102 (SEQ ID NO: 2) antisense oligonucleotide is 30-nt long and a literature survey suggests that long molecules tend to form dimers once introduced into cells. Interaction with an RNA molecule is a necessary step if the antisense oligonucleotide is to inhibit translation of the targeted RNA molecule thereby lowering the levels of IL-15 production. Therefore, truncated versions of HCL-102 (SEQ ID NO: 2) were designed. Oligonucleotides truncated from the 5′ end were designated HCL-109 (HCL-102 L1; SEQ ID NO: 10), HCL-110 (HCL-102 L2; SEQ ID NO: 11) and HCL-111 (HCL-102 L3; SEQ ID NO: 12) and are 27, 24 and 21 oligonucleotides in length, respectively. Oligonucleotides truncated from the 3′ end were designated HCL-112 (HCL-102 R1; SEQ ID NO: 13), HCL-113 (HCL-102 R2; SEQ ID NO: 14) and HCL-114 (HCL-102 R3; SEQ ID NO: 15) and are 24, 21 and 18 oligonucleotides in length, respectively. These antisense oligonucleotides reproducibly inhibited IL-15 expression in CV-1/EBNA cells (see FIGS. 11 and 12). HCL-102 L2 (SEQ ID NO: 11) inhibits IL-15 production to about 55% of control activity at concentrations as low as 0.1 μM. In addition, HCL-102 R2 (SEQ ID NO: 14) inhibits IL-15 production to about 16% of control activity at similar concentrations. Another effective oligonucleotide from the group is HCL-103 which has shown inhibitory effect in a reproducible manner (FIG. 5).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

What is claimed is:

1. A method for ameliorating an IL-15-associated disorder in a subject, comprising administering to the subject having an IL-15-associated disorder, a therapeutically effective amount of a composition containing an antisense oligonucleotide, wherein said oligonucleotide interacts with a polynucleotide encoding IL-15 thereby inhibiting IL-15 production.

2. The method of claim 1, wherein the antisense oligonucleotide is expressed from an expression vector.

3. The method of claim 2, wherein the vector is a plasmid.

4. The method of claim 2, wherein the vector is a viral vector.

5. The method of claim 1, wherein the IL-15-associated disorder is selected from the group consisting of inflammatory bowel disease, arthritis, cirrhosis, multiple sclerosis, chronic liver disease, ulcerative colitis and cell proliferative disorders.

6. The method of claim 1, wherein the antisense oligonucleotide is from about 8 to 40 nucleic acids in length.

7. The method of claim 1, wherein the antisense oligonucleotide is chemically modified.

8. The method of claim 7, wherein the chemical modification is by substitution in a non-bridging oxygen atom of the antisense nucleic acid back bone with a moiety selected from the group consisting of methane phosphate, methyl phosphate, phosphoramidite, and phosphorthioate.

9. The method of claim 8, wherein the substitution is at the 5′ terminal region or the 3′ terminal region.

10. The method of claim 1, wherein the antisense oligonucleotide is DNA.

11. The method of claim 1, wherein the antisense oligonucleotide is RNA.

12. The method of claim 1, wherein the antisense oligonucleotide is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO. 7, SEQ ID NO: 8, SEQ ID NO: 10, SEQ ID NO: 11, SEQ ID NO: 12, SEQ ID NO: 13, SEQ ID NO: 14, SEQ ID NO: 15 and any combination thereof.

13. The method of claim 1, wherein the subject is a mammal.

14. The method of claim 13, wherein the mammal is a human.

15. The method of claim 1, wherein the polynucleotide encoding IL-15 is DNA.

16. The method of claim 1, wherein the polynucleotide encoding IL-15 is RNA.

17. The method of claim 16, wherein the RNA is mRNA.

18. A method of inhibiting production of IL-15 in vivo, comprising administering to a subject having an IL-15-associated disorder, a therapeutically effective amount of a composition containing an antisense oligonucleotide, wherein said oligonucleotide interacts with a polynucleotide encoding IL-15 thereby inhibiting IL-15 production.

19. The method of claim 18, wherein the antisense oligonucleotide is expressed from an expression vector.

20. The method of claim 19, wherein the vector is a plasmid.

21. The method of claim 19, wherein the vector is a viral vector.

22. The method of claim 18, wherein the IL-15-associated disorder is selected from the group consisting of inflammatory bowel disease, arthritis, cirrhosis, multiple sclerosis, chronic liver disease, ulcerative colitis and cell proliferative disorders.

23. The method of claim 18, wherein the antisense oligonucleotide is from about 8 to 40 nucleic acids in length.

24. The method of claim 18, wherein the antisense oligonucleotide is chemically modified.

25. The method of claim 24, wherein the chemical modification is by substitution in a non-bridging oxygen atom of the antisense nucleic acid back bone with a moiety selected from the group consisting of methane phosphate, methyl phosphate, phosphoramidite, and phosphorthioate.

26. The method of claim 25, wherein the substitution is at the 5′ terminal region or the 3′ terminal region.

27. The method of claim 18, wherein the antisense oligonucleotide is DNA.

28. The method of claim 18, wherein the antisense oligonucleotide is RNA.

29. The method of claim 18, wherein the antisense oligonucleotide is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO. 7, SEQ ID NO: 8, SEQ ID NO: 10, SEQ ID NO: 11, SEQ ID NO: 12, SEQ ID NO: 13, SEQ ID NO: 14, SEQ ID NO: 15, and any combination thereof.

30. The method of claim 18, wherein the subject is a mammal.

31. The method of claim 30, wherein the mammal is a human.

32. A method of inhibiting production of IL-15, comprising contacting a sample containing a polynucleotide encoding IL-15 with an inhibiting effective amount of IL-15 antisense oligonucleotide.

33. The method of claim 32, wherein the antisense oligonucleotide is in an expression vector.

34. The method of claim 33, wherein the vector is a plasmid.

35. The method of claim 33, wherein the vector is a viral vector.

36. The method of claim 32, wherein the antisense oligonucleotide is from about 8 to 40 nucleic acids in length.

37. The method of claim 32, wherein the antisense oligonucleotide is chemically modified.

38. The method of claim 37, wherein the chemical modification is by substitution in a non-bridging oxygen atom of the antisense nucleic acid back bone with a moiety selected from the group consisting of methane phosphate, methyl phosphate, phosphoramidite, and phosphorthioate.

39. The method of claim 38, wherein the substitution is at the 5′ terminal region or the 3′ terminal region.

40. The method of claim 32, wherein the antisense oligonucleotide is DNA.

41. The method of claim 32, wherein the antisense oligonucleotide is RNA.

42. The method of claim 32, wherein the antisense oligonucleotide is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO. 7, SEQ ID NO: 8, SEQ ID NO: 10, SEQ ID NO: 11, SEQ ID NO: 12, SEQ ID NO: 13, SEQ ID NO: 14, SEQ ID NO: 15, and any combination thereof.

43. The method of claim 32, wherein the sample contains cells.

44. The method of claim 32, wherein the sample is a tissue.

45. An antisense oligonucleotide about 8 to 40 nucleic acids in length comprising a contiguous nucleic acid sequence which selectively binds to an IL-15 polynucleotide.

46. The antisense oligonucleotide of claim 45, wherein said antisense oligonucleotide is chemically modified by a substitution in a non-bridging oxygen atom of the antisense nucleic acid back bone with a moiety selected from the group consisting of methane phosphate, methyl phosphate, phosphoramidite, and phosphorthioate.

47. The antisense oligonucleotide of claim 45, wherein the substitution is at the 5′ terminal region or the 3′ terminal region.

48. The antisense oligonucleotide of claim 45, wherein the antisense oligonucleotide is DNA.

49. The antisense oligonucleotide of claim 45, wherein the antisense oligonucleotide is RNA.

50. The antisense oligonucleotide of claim 45, wherein the antisense oligonucleotide is contained in a vector.

51. The antisense oligonucleotide of claim 50, wherein the vector is an expression vector.

52. The antisense oligonucleotide of claim 50, wherein the vector is a plasmid.

53. The antisense oligonucleotide of claim 50, wherein the vector is a viral vector.

54. The antisense oligonucleotide of claim 45, wherein the antisense oligonucleotide is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO. 7, SEQ ID NO: 8, SEQ ID NO: 10, SEQ ID NO: 11, SEQ ID NO: 12, SEQ ID NO: 13, SEQ ID NO: 14, SEQ ID NO: 15, and any combination thereof.

55. An antisense oligonucleotide complementary to a polynucleotide encoding IL-15 and which hybridizes to a nucleic acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO. 7, SEQ ID NO: 8, SEQ ID NO: 10, SEQ ID NO: 11, SEQ ID NO: 12, SEQ ID NO: 13, SEQ ID NO: 14, and SEQ ID NO: 15.

56. A recombinant nucleic acid sequence which, upon transcription, provides an antisense oligonucleotide, wherein the oligonucleotide modulates expression of IL-15 by interacting with a polynucleotide encoding IL-15.

57. The recombinant nucleic acid sequence of claim 56, wherein the polynucleotide encoding IL-15 is DNA.

58. The recombinant nucleic acid sequence of claim 56, wherein the polynucleotide encoding IL-15 is RNA.

59. The recombinant nucleic acid sequence of claim 56, wherein the modulating of IL-15 expression is by inhibition.

60. A pharmaceutical composition comprising an antisense oligonucleotide selected from the group consisting of SEQ ID NO: 1, SEQ ID NO: 2, SEQ ID NO: 3, SEQ ID NO: 4, SEQ ID NO: 5, SEQ ID NO: 6, SEQ ID NO. 7, SEQ ID NO: 8, SEQ ID NO: 10, SEQ ID NO: 11, SEQ ID NO: 12, SEQ ID NO: 13, SEQ ID NO: 14, SEQ ID NO: 15, and any combination thereof.

61. A method of monitoring the effectiveness of suppressing IL-15 production after administering a therapeutically effective amount of the antisense oligonucleotide of claim 43, comprising detecting the level of IL-15 production in a sample before and after the antisense therapy.
